(19) United States
(12) Patent Application Publication
(10) Pub. No.: US 2003/0099604 A1
(43) Pub. Date: May 29, 2003

(54) Lipstick composition

(76) Inventor: Orrea Light, New York, NY (US)

Correspondence Address: KATTEN MUCHIN ZAVIS ROSENMAN, 575 MADISON AVENUE, NEW YORK, NY, 10022-2585, US

(21) Appl. No.: 10/299,392

(22) Filed: Nov. 19, 2002

Related U.S. Application Data
(60) Provisional application No. 60/331,684, filed on Nov. 19, 2001.

(51) Int. Cl. A61K 7/025; A61K 7/06

(52) U.S. Cl. 424/64; 424/74

(57) ABSTRACT

A lipstick composition that overcomes the dry and waxy feel of lipstick, that applies softly, does not drag upon application, and leaves the wearers lips feeling comfortable and protected against cracking and dryness.

### CROSS-REFERENCE TO RELATED APPLICATION

This application claims priority under 35 U.S.C. §119(e) from U.S. Provisional Patent application No.60/331,684 filed on Nov. 19, 2001, and which is incorporated herein by reference.

### FIELD OF THE INVENTION

This invention relates to a lipstick composition, and more particularly to a lipstick composition that imparts a beneficial moisturizing effect, is easily applied on the lips, spreads smoothly and imparts increased cushion and improved lip comfort.

### BACKGROUND OF INVENTION

Lip appearance has been enhanced by the use of colorants since antiquity. Artifacts from early Egyptian, Babylonian and Sumerian civilizations disclose that women in these societies painted their lips with mixtures of hematite and red ochre in animal fat or vegetable oil. Lip cosmetics were used by Syrians, Persians, Greeks and Romans for aesthetic, medical or ritualistic purposes.

Lipsticks have been used for many years to accentuate the positive aspects of the wearer's lips. Lipsticks are capable of altering the apparent facial characteristics of the wearer; narrow lips may be widened and broad lips narrowed. Besides altering the shape of the lips, lipsticks can be made in a great number of colors and shades to promote a desired effect or express the mood of the wearer.

Color imparted to the lips helps to define the mouth area while imparting cosmetic shades that are suitable with fashion trends. The color is ordinarily provided by insoluble pigments such as lakes of dye finely dispersed in the oily vehicle and one or more fluorescein dye derivatives that serve to stain the lips. When dyes are used, a solvent for the dye is also included for increasing the effectiveness of this staining on the lips.

Lipsticks, in general, are made of an oily vehicle comprising fat or oil stiffened to a desired consistency with waxes of various types, which serve to raise the melting point and improve the physical stability. Modem lipsticks comprise a base of oil and wax and one or several dissolved or suspended colorants. These lipstick formulations first appeared in the early years of the twentieth century. Carmine was the original lip colorant of choice replaced in the late 1920s by eosin. Later fluorescein-based stains followed, until today a plethora of colors and finishes are available, ranging from opaque full-coverage lipsticks to sheer or colorless lip glosses, all available in a choice of stick or paint vehicles.

Contemporary lipsticks may be classified by primary colorant (stain or pigment), appearance (cream or pearl) or finish on lips (matte or glossy). Lipstick wax bases may also be categorized by chemical class (organic, silicone), source (natural, synthetic), function (moisturizing, contouring) and so on.

Molded lipsticks comprise a solid fatty base containing dissolved and suspended dyes, preservatives and fragrance in admixture with cosmetically acceptable waxes, oils, solids and semisolids. Waxy and oily materials are included in lipsticks to give the lips a moist and alluring look. Several of the most important materials used in lipstick compositions are cosmetic waxes such as beeswax, Candelilla wax, Carnauba wax and ozokerite and cosmetic oils such as castor oil and lanolin. Beeswax adds binding and molding properties to lipstick, Candellila wax gives lipstick hardness, rigidity and high gloss and Carnauba wax and ozokerite give molded lipstick toughness; castor oil is a solvent for the dyes and functions as an emollient and lanolin aids in maintaining homogeneity during manufacturing as well as serving as an emollient. Emollients provide a supple and pleasant feeling to the lips of the wearer. Dyes, preservative and fragrance are typically also present.

Lipsticks that contain water in addition to waxes and oils purportedly are more effective in moisturizing the lips. Despite the purported benefits of water-containing lipsticks, consumers still favor anhydrous lipsticks since these usually last much longer. Among the other numerous materials useful for incorporation into a lipstick composition, fatty alcohols and fatty acid esters have been found to be useful in cosmetic products because of the ability of such compounds to maintain a porous fatty film on the lips.

Lips typically have a rough surface comprising tiny ridges and cracks. Such cracks detract from the smooth elegant appearance desired by most wearers. It is desirable to provide a lipstick composition that fills these the tiny crevices on the surface of the lip and gives the wearer's lips a smoother more even surface. In addition, the small cracks, ridges and fissures typical are the initiation point of lip cracking and damage during dry and cold conditions.

Thus, in addition to the cosmetic benefits of filling these tiny ridges and cracks to provide a smoother more elegant appearing look and feel, there exists the need to provide a lipstick ridge and crack filling composition which provides a method of forestalling the presence of these ridges and cracks from acting as the nucleus of cracks and breakages of the skin during cold or dry weather.

### SUMMARY OF INVENTION

The present invention is a lipstick formulation which imparts a beneficial moisturizing effect, is easily applied on the lips, spreads smoothly and imparts increased cushion providing a unique feel to the wearers lips while at the same time providing added protection against adverse environmental conditions.

This lipstick imparts these properties by utilizing a gel/cream base incorporating a mixture of canola oil, glycine soja (soybean) germ extract, zea mays (corn) starch and silica and two botanicals as a synergistic complex to aide in moisturization. A significant part of the composition is the use of a base system comprising the mixture of canola oil, glycine soja (soybean) germ extract, zea mays (corn) starch and silica to provide “extra cushion” and soft rich feel to the lipstick.

### BRIEF DESCRIPTION OF THE DRAWINGS

FIG. 1 is a table of the ingredients of compositions within the scope of the present invention.

### DETAILED DESCRIPTION OF THE INVENTION

Lips typically have a rough surface comprising tiny ridges and cracks. It is desirable to provide a lipstick composition that fills these the tiny crevices on the surface of the lip and gives the wearer's lips a smoother more even surface. A lipstick base having high cushion gels incorporated into the product allows the product to fill in the rough areas. The high cushion gels in the disclosed composition, in combination with the other ingredients, deposit a thicker film on the lips and provide a smoother, more harmonious surface to the wearer's lips.

The disclosed lipstick composition provides a unique feel and ease in application. It imparts cushion, creaminess, moisturization and substantivity (thicker film on lip) to the wearer thereby providing increased comfort and ease in application to the consumer. This lipstick imparts these properties by utilizing a gel/cream base incorporating a mixture of canola oil, glycine soja (soybean) germ extract, zea mays (corn) starch and silica and by utilizing two botanicals as a synergistic complex to aide in moisturization. A significant part of the composition is the use of a base system comprising the mixture of canola oil, glycine soja (soybean) germ extract, zea mays (corn) starch and silica to provide “extra cushion” and soft rich feel to the lipstick.

The composition utilizes a gel/cream base with incorporates canola gel, a proprietary mixture of canola oil, glycine soja (soybean) germ extract, zea mays (corn) starch and silica sold by Natunola Health (Nepean, Ontario, Canada) under its trademark Vegelatum® Equiline.

Canola Oil is a vegetable oil from the canola seed that is very stable, high in oleic acid, rich in Vitamin E that is an excellent emollient and moisturizer and helps reduce skin irritation. Vegelatum® Equiline is a botanical emollient produced from a non-transgenically modified canola oil.

Canola gel is superior to petroleum based emollients, such as petrolatum, as it has exceptional thermostability, a gel-like structure and is also less greasy. In combination with the other ingredients of the gel/cream base, it softens and smoothes the skin, forming a light film which prevents evaporation of moisture from the skin and protects the skin from irritation.

In appearance canola gel is an off-white, opaque gel, with a very light odour. It is soluble in all vegetable oils, glycerol triisostearate, isostearyl isosterate, oleic acid, isostearic acid, coco-caprylate/caprate and mixed glycerides and insoluble in water, absolute ethanol and 1,2-propanadiol, dimethicone and cyclomethicone.

The base also incorporates Euphorbia cerifera wax (Candelilla wax), a natural vegetable wax used as a film former and for skin conditioning.

The composition also includes one or more oils or oil-like emollients. Any cosmetically or pharmaceutically acceptable oil may be used in the wax base, the selection only being limited by the necessity for successfully wetting out pigments, a technique well known in the art. Examples of suitable oils or oil-like emollients can be found in the International Cosmetic Ingredient Handbook, CTFA, 1996, the contents of which are incorporated herein by reference.

Useful materials include, but are not limited to, castor oil, coconut oil, corn oil, jojoba oil, cottonseed oil, soybean oil, walnut oil, wheat germ oil, sunflower seed oil, palm kernel oil, calendula oil, C8-18 triglycerides, lanolin and lanolin derivatives, illipe butter, shea butter; esters, such as isodecyl neopentanoate, tridecyl octanoate, diisostearyl malate, cetyl palmitate, cetyl octanoate, cetyl stearate, cetyl myristate, isopropyl palmitate, isopropyl myristate, dipentaerythrityl hexahydroxy stearate/stearate/rosinate, polyglyceryl-2-isostearate, neopentyl glycol distearate, isodecyl oleate, decyl isostearate, diisopropyl sebacate, PEG-4 diheptanoate, dioctyl malate, and isohexyl neopentanoate; fatty alcohols, such as lanolin alcohol or oleyl alcohol; and silicone oils, such as cyclomethicone, dimethicone, cetyl dimethicone, lauryl trimethicone, and dimethiconol.

Compositions of the present invention contain sufficient pigments to provide the look sought by the user. The amount of pigment present in the composition is in the range of 0 to approximately 25% by weight of the total composition, preferably from about 0 present to approximately 18% by weight of the total composition.

Pigments which may be used herein are all inorganic and organic colors/pigments suitable for use in lip composition compositions. These are usually aluminum, barium or calcium salts or lakes. Lakes are either a pigment that is extended or reduced with a solid diluent or an organic pigment that is prepared by the precipitation of a water-soluble dye on an adsorptive surface, which usually is aluminum hydrate. A lake also forms from precipitation of an insoluble salt from an acid or basic dye. Calcium and barium lakes are also used herein.

Preferred lakes of the present invention are Red 3 Aluminum Lake, Red 21 Aluminum Lake, Red 27 Aluminum Lake, Red 28 Aluminum Lake, Red 33 Aluminum Lake, Yellow 5 Aluminum Lake, Yellow 6 Aluminum Lake, Yellow 10 Aluminum Lake, Orange 5 Aluminum Lake and Blue 1 Aluminum Lake, Red 6 Barium Lake, Red 7 Calcium Lake. Other colors and pigments can also be included in the lip compositions, such as pearls, titanium oxides, Red 6, Red 21, Blue 1, Orange 5, and Green 5 dyes, chalk, talc, iron oxides and titanated micas.

There are a number of other ingredients approved for use in the cosmetic art that may be used in compositions of the present invention. Such ingredients are those approved for use in cosmetics and can be found listed in reference books such as the CTFA Cosmetic Ingredient Handbook, Second Edition, The Cosmetic, Toiletries, and Fragrance Association, Inc. 1988, 1992. Said materials may be used provided their inclusion does not significantly disrupt the composition once it has been applied to the skin wherein a film has been formed. Said ingredients include waxes, fragrances, flavor oils, skin care ingredients such as sunscreen, emulsifiers and the like. Hypoallergenic compositions can be made into the present invention where said compositions do not contain fragrances, flavor oils, lanolin, sunscreens, particularly PABA, or other sensitizers and irritants.

Waxes used in the present invention are used at levels that do not interfere with film formation process. Generally waxes are not used in the present invention higher than about 12.5% of the composition, preferably not higher than about 8% of the composition. Waxes are defined as lower-melting organic mixtures or compounds of high molecular weight, solid at room temperature and generally similar in composition to fats and oils except that they contain no glycerides. Some are hydrocarbons, others are esters of fatty acids and alcohols. Waxes useful in the present invention are selected from the group consisting of animal waxes, vegetable waxes, mineral waxes, various fractions of natural waxes, synthetic waxes, petroleum waxes, ethylenic polymers, hydrocarbon types such as Fischer-Tropsch waxes, silicone waxes, and mixtures thereof. Waxes preferred for use in the present invention are selected from the group consisting of Euphorbia cerifera (Candelilla) wax; microcrystalline wax; Ozokerite and paraffin. The waxes most useful herein are the Ozokerite waxes.

Flavor oils such as peppermint oil, orange oil, citrus oil, or wintergreen oil can be used along with an alcohol or glycerine. Flavor oils are usually mixed in a solvent such as ethanol to dilute the flavor. The flavor oils useful herein can be derived from natural sources or be synthetically prepared. Generally, flavor oils are mixtures of ketones, alcohols, fatty acids, esters and terpenes. The term “flavor oil” is generally recognized in the art to be a liquid which is derived from botanical sources, i.e. leaves, bark, or skin of fruits or vegetables, and which are usually insoluble in water. The level of flavor oil used can range from 0% to about 0.5%, preferably from 0% to about 0.3% of the lip composition.

Emulsifiers may be used as coupling agents that have an affinity for the hydrophilic and hydrophobic phases of lip compositions of this invention. Such emulsifiers include those routinely used in cosmetics and are found in the CTFA.

Skin care active ingredients in both water-soluble and water insoluble forms can be added to the lip composition. Said ingredients may include fat-soluble vitamins, sunscreens and pharmaceutically active ingredients. Skin care active ingredients include glycerine, zinc oxide; chamomile oil; ginko biloba extract; pyroglutamic acid, salts or esters; sodium hyaluronate; 2-hydroxyoctanoic acid; sulfur; salicylic acid; carboxymethyl cysteine, water, propylene glycol and mixtures thereof.

The most important parts of the composition and those which provide the superior qualities are the gels in the base, the lauroyl lysine and talc, and the synergistic combination of Tamanol and Prunus oil. Tamanol and Prunus oil both aid in hydration and dryness prevention. Prunus oil is further recognized scientifically as a free-radical scavenger. Tamanol's beneficial effects of tissue regeneration and healing are associated with a polyunsaturated fatty acid, calophyllic acid.

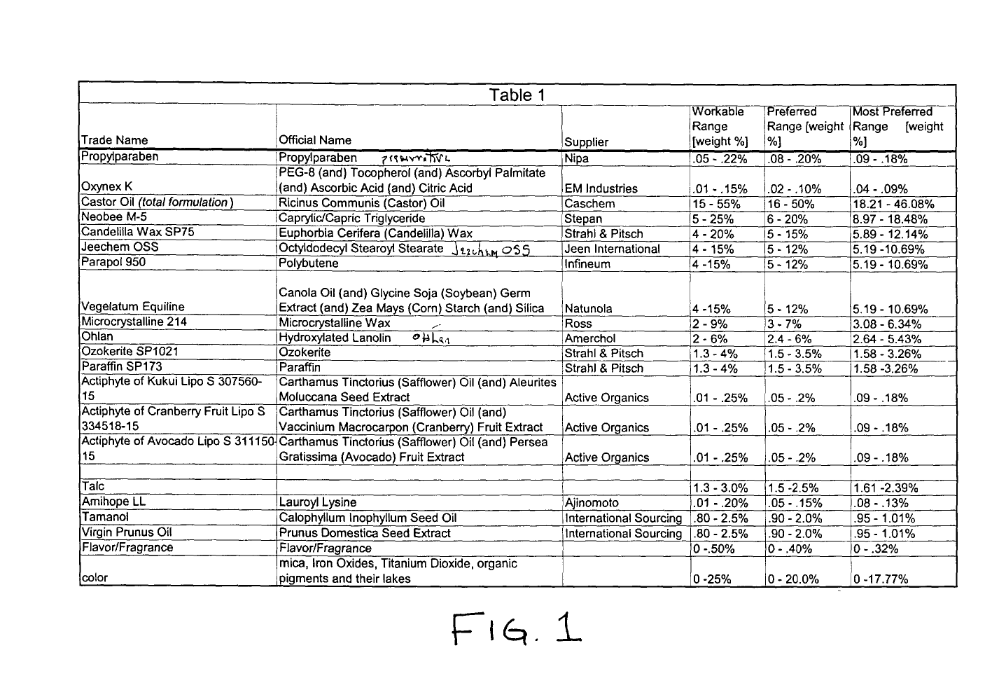

It is the use of these ingredients, in the specified proportions and alone or in combination with the other listed ingredients, all as set forth in the table of FIG. 1, that yields the superior results obtained. Table 1 describes the ingredients most useful in compositions of this invention along with preferred embodiments of the inventive composition.

In a most preferred embodiment, the disclosed lipstick compositions imparting increased cushion, creaminess, moisturization and substantivity comprise propylparaben; PEG-8 (and) tocopherol (and) ascorbyl palmitate (and) ascorbic acid (and) citric acid; Ricinus communis (castor) oil; caprylic/capric triglyceride; Euphorbia cerifera (candelilla) wax; octyldodecyl stearoyl stearate; Polybutene; canola oil (and) glycine soja (soybean) germ extract (and) Zea mays (corn) starch (and) silica; microcrystalline wax; hydroxylated lanolin; ozokerite; paraffin; Carthamus tinctorius (safflower) oil (and) aleurites moluccana seed extract; Vaccinium macrocarpon (cranberry) fruit extract; Persea gratissima (avocado) fruit extract; talc; lauroyl lysine; Calophyllum inophyllum seed oil; Prunus domestica seed extract; flavor/fragrance; mica, iron oxides, titanium dioxide, organic pigments and their lakes.

I claim:

1. A lipstick composition imparting increased cushion, creaminess, moisturization and substantivity comprising vegelatum equiline, tamanol and virgin prunus oil.

2. The lipstick composition of claim I comprising vegelatum equiline in an amount of from about 4 to about 15 weight %, tamanol in an amount of from about 0.8 to about 2.5 weight %, and virgin prunus oil in an amount of from about 0.8 to about 2.5 weight %.

3. The lipstick composition of claim 2 comprising vegelatum equiline in an amount of from about 5 to about 12 weight %, tamanol in an amount of from about 0.9 to about 2 weight %, and virgin prunus oil in an amount of from about 0.9 to about 2 weight %.

4. The lipstick composition of claim 2 further comprising talc in an amount of from about 1.3 to about 3 weight %, and lauroyl lysine in an amount of from about 0.01 to about 0.20 weight %.

5. The lipstick composition of claim 2 further comprising talc in an amount of from about 1.5 to about 2.5 weight %, and lauroyl lysine in an amount of from about 0.05 to about 0.15 weight %.

6. A lipstick composition imparting increased cushion, creaminess, moisturization and substantivity comprising propylparaben; PEG-8 (and) tocopherol (and) ascorbyl palmitate (and) ascorbic acid (and) citric acid; Ricinus communis (castor) oil; caprylic/capric triglyceride; Euphorbia cerifera (candelilla) wax; octyldodecyl stearoyl stearate; Polybutene; canola oil (and) glycine soja (soybean) germ extract (and) Zea mays (corn) starch (and) silica; microcrystalline wax; hydroxylated lanolin; ozokerite; paraffin; Carthamus tinctorius (safflower) oil (and) aleurites moluccana seed extract; Vaccinium macrocarpon (cranberry) fruit extract; Persea gratissima (avocado) fruit extract; talc; lauroyl lysine; Calophyllum inophyllum seed oil; Prunus domestica seed extract; flavor/fragrance; mica, iron oxides, titanium dioxide, organic pigments and their lakes.

7. The lipstick composition of claim 6 additionally comprising fragrance and/or flavor components.

8. The lipstick composition of claim 6 comprising

| | Minimum | Maximum |
|---|---|---|
| Ingredient | [weight %] | [weight %] |
| Propylparaben | 0.05 | 0.22 |
| PEG-8/Tocopherol/Ascorbyl Palmitate/ | 0.01 | 0.15 |
| Ascorbic Acid (and) Citric Acid | | |
| Ricinus Communis (Castor) Oil | 15 | 55 |
| Caprylic/Capric Triglyceride | 5 | 25 |
| Euphorbia Cerifera (Candelilla) Wax | 4 | 20 |
| Octyldodecyl Stearoyl Stearate | 4 | 15 |
| Polybutene | 4 | 15 |
| Canola Oil/Glycine Soja (Soybean) Germ | 4 | 15 |
| Extract/Zea Mays (Corn) Starch/Silica | | |
| Microcrystalline Wax | 2 | 9 |
| Hydroxylated Lanolin | 2 | 6 |
| Ozokerite | 1.3 | 4 |
| Paraffin | 1.3 | 4 |
| Carthamus Tinctorius (Safflower) Oil/ | 0.01 | 0.25 |
| Aleurites Moluccana Seed Extract | | |
| Carthamus Tinctorius (Safflower) Oil/ | 0.01 | 0.25 |
| Vaccinium Macrocarpon (Cranberry) Fruit | | |
| Extract | | |
| Carthamus Tinctorius (Safflower) Oil/ | 0.01 | 0.25 |
| Persea Gratissima (Avocado) Fruit Extract | | |
| Talc | 1.3 | 3 |
| Lauroyl Lysine | 0.01 | 0.20 |
| Calophyllum Inophyllum Seed Oil | 0.80 | 2.5 |
| Prunus Domestica Seed Extract | 0.80 | 2.5 |
| Flavor/Fragrance | 0 | 0.50 |
| mica, Iron Oxides, Titanium Dioxide, | 0 | 25 |
| organic pigments and their lakes | | |

9. The lipstick composition of claim 6 comprising

| | Minimum | Maximum |
|---|---|---|
| Ingredient | [weight %] | [weight %] |
| Propylparaben | 0.08 | 0.20 |
| PEG-8/Tocopherol/Ascorbyl Palmitate/ | 0.02 | 0.10 |
| Ascorbic Acid (and) Citric Acid | | |
| Ricinus Communis (Castor) Oil | 16 | 50 |
| Caprylic/Capric Triglyceride | 6 | |
| Euphorbia Cerifera (Candelilla) Wax | 5 | 15 |
| Octyldodecyl Stearoyl Stearate | 5 | 12 |
| Polybutene | 5 | 12 |
| Canola Oil/Glycine Soja (Soybean) Germ | 5 | 12 |
| Extract/Zea Mays (Corn) Starch/Silica | | |
| Microcrystalline Wax | 3 | 7 |
| Hydroxylated Lanolin | 2.4 | 6 |
| Ozokerite | 1.5 | 3.5 |
| Paraffin | 1.5 | 3.5 |
| Carthamus Tinctorius (Safflower) Oil/ | 0.05 | 0.2 |
| Aleurites Moluccana Seed Extract | | |
| Carthamus Tinctorius (Safflower) Oil/ | 0.05 | 0.2 |
| Vaccinium Macrocarpon (Cranberry) Fruit | | |
| Extract | | |
| Carthamus Tinctorius (Safflower) Oil/ | 0.05 | 0.2 |
| Persea Gratissima (Avocado) Fruit | | |
| Extract | | |
| Talc | 1.5 | 2.5 |
| Lauroyl Lysine | 0.05 | 0.15 |
| Calophyllum Inophyllum Seed Oil | 0.90 | 2 |
| Prunus Domestica Seed Extract | 0.90 | 2 |
| Flavor/Fragrance | 0 | 40 |
| mica, Iron Oxides, Titanium Dioxide, | 0 | 20 |
| organic pigments and their lakes | | |

10. The lipstick composition of claim 6 comprising

| | Minimum | Maximum |
|---|---|---|
| Ingredient | [weight %] | [weight %] |
| Propylparaben | 0.09 | 0.18 |
| PEG-8/Tocopherol/Ascorbyl Palmitate/ | 0.04 | 0.09 |
| Ascorbic Acid (and) Citric Acid | | |
| Ricinus Communis (Castor) Oil | 18.21 | 46.08 |
| Euphorbia Cerifera (Candelilla) Wax | 5.89 | 12.14 |
| Octyldodecyl Stearoyl Stearate | 5.19 | 10.69 |
| Polybutene | 5.19 | 10.69 |
| Canola Oil/Glycine Soja (Soybean) Germ | 5.19 | 10.69 |
| Extract/Zea Mays (Corn) Starch/Silica | | |
| Microcrystalline Wax | 3.08 | 6.34 |
| Hydroxylated Lanolin | 2.64 | 5.43 |
| Ozokerite | 1.58 | 3.26 |
| Paraffin | 1.58 | 3.26 |
| Carthamus Tinctorius (Safflower) Oil/ | 0.09 | 0.18 |
| Aleurites Moluccana Seed Extract | | |
| Carthamus Tinctorius (Safflower) Oil/ | 0.09 | 0.18 |
| Vaccinium Macrocarpon (Cranberry) Fruit | | |
| Extract | | |
| Carthamus Tinctorius (Safflower) Oil/ | 0.09 | 0.18 |
| Persea Gratissima (Avocado) Fruit | | |
| Extract | | |
| Talc | 1.61 | 2.39 |
| Lauroyl Lysine | 0.08 | 0.13 |
| Calophyllum Inophyllum Seed Oil | 0.95 | 1.01 |
| Prunus Domestica Seed Extract | 0.95 | 1.01 |
| Flavor/Fragrance | 0 | 0.32 |
| mica, Iron Oxides, Titanium Dioxide, | 0 | 17.77 |
| organic pigments and their lakes | | |
